SEQUENCE LISTING (11 sequences)

SEQ ID NO: 1 - DNA, 882 bp, Escherichia coli
  CDS (1)..(882)
atgacacgacaaaaagcaacgctcatagggctgatagcgatcgtcctgtggagcacgatggtaggattgattcgcggtgtcagtgaggggctcggcccggtcggcggcgcagctgctatctattcattaagcgggctgctgttaatcttcacggttggatttccgcgtattcggcaaatcccgaaaggctatttactcgccgggagtctgttattcgtcagctatgaaatctgtctggcgctttccttagggtatgcggcgacccatcatcaggcgattgaagtgggtatggtgaactatctgtggcccagcctgacaattctctttgccattctgtttaatggtcagaaaaccaactggttgattgtacctggattattattagccctcgtcggcgtctgttgggtgttaggcggtgacaatgggttacattatgatgaaatcatcaataatatcaccaccagcccattgagttatttcctggcgttcattggtgcgtttatctgggcagcctattgcacagtaacgaataaatacgcacgcggatttaatggaattaccgtttttgtcctgctaacgggagcaagtctgtgggtttactattttcttacgccacaaccagaaatgatatttagcacgcccgtcatgattaaactcatctctgcggcatttaccttaggatttgcttatgctgcatggaatgtcggtatattgcatggcaatgtcaccattatggcggtaggttcgtattttacgcctgtactttcctcagcgcttgcagccgtgctgctcagcgccccgctgtcgttctcgttctggcaaggcgcgctgatggtctgcggcggttccctgctctgctggctggcgacacgtcgtggttaa

SEQ ID NO: 2 - protein, 293 aa, Escherichia coli
MTRQKATLIGLIAIVLWSTMVGLIRGVSEGLGPVGGAAAIYSLSGLLLIFTVGFPRIRQIPKGYLLAGSLLFVSYEICLALSLGYAATHHQAIEVGMVNYLWPSLTILFAILFNGQKTNWLIVPGLLLALVGVCWVLGGDNGLHYDEIINNITTSPLSYFLAFIGAFIWAAYCTVTNKYARGFNGITVFVLLTGASLWVYYFLTPQPEMIFSTPVMIKLISAAFTLGFAYAAWNVGILHGNVTIMAVGSYFTPVLSSALAAVLLSAPLSFSFWQGALMVCGGSLLCWLATRRG

SEQ ID NO: 3 - DNA, 291 bp, Escherichia coli
  CDS (1)..(291)
atgaaattaaaaatagttgcggtggttgtaactggtttgttagctgcgaacgtagcacacgctgccgaagtctataacaaggatggtaataaactcgacctttatggcaaggttaccgctctacgttattttactgatgataagcgtgacgatggtgataaaacttatgcccgtctcggctttaaaggagaaacgcaaatcaatgatcaaatgattggttttggtcactgggaatatgattttaaaggctataacgatgaagccaacggctcgcgcggcaagaatctctga

SEQ ID NO: 4 - protein, 96 aa, Escherichia coli
MKLKIVAVVVTGLLAANVAHAAEVYNKDGNKLDLYGKVTALRYFTDDKRDDGDKTYARLGFKGETQINDQMIGFGHWEYDFKGYNDEANGSRGKNL

SEQ ID NO: 5 - DNA, 957 bp, Escherichia coli
  CDS (1)..(957)
atgatcactgaccttatattacacaatcatcccaggatgaaaacaatcactttaaacgacaaccatattgcacatttaaacgccaaaaacactacaaaactggaatatttaaacttaagcaataacaatttactgccaaccaatgacattgatcaactaatatcatcaaaacatctttggcatgtattagttaacggcatcaacaatgatccacttgcacaaatgcagtactggactgcagtaagaaatataattgatgacactaatgaagtgaccattgatttatcaggacttaatttaaccactcaaccaccagggctgcaaaacttcacatctatcaatcttgataataaccaattcacacattttgatgcaaccaactacgatagactcgtaaagctaagtctgaatagtaatgctcttgagtcaataaattttcctcaaggcagaaatgtaagtattacacatatatctatgaataataatgctctcagaaatattgatatagataggctttcatcagttacttattttagtgcggcacataatcaactagagtttgtgcaattagaatcttgcgaatggctgcaatacctgaatctcagccataatcaattaactgatattgttgcaggtaataaaaatgaactcttactgctggatctatcccataataaactaacaagtttacacaatgacttatttcccaacttgaatacgttacttattaacaacaatttgctttctgaaattaaaatattctatagcaacttctgcaatgttcagacattaaacgctgctaacaaccagttgaaatatataaatcttgatttcctgacttatcttccatctatcaaaagtttaagactggacaataataaaataacccacattgatactaataatacatccgatattggaactttattccccataataaaacagagcaaaacttaa

SEQ ID NO: 6 - protein, 318 aa, Escherichia coli [sequence omitted]

SEQ ID NO: 7 - DNA, 336 bp, Escherichia coli
  CDS (1)..(336)
atgcagctcatgttatttaatttgttttccccagcacttaagcttaatactggcctggcaattctttcgcctggtgcatttgaagttcactctgacggaatagatgtggataacgaattgtttcactatcctattaaaaaagcatataccccatataatatacacacttacaagacagaggaagttgtaaaccagatgaatataaaagttaaaaacatgaccttagatgaaataaacaatacttactgtaataacgattattacaatcaggcaataagagaggaaccgatagaccttctggacagatcgttttcctccagttcatggcctttttag

SEQ ID NO: 8 - protein, 111 aa, Escherichia coli
MQLMLFNLFSPALKLNTGLAILSPGAFEVHSDGIDVDNELFHYPIKKAYTPYNIHTYKTEEVVNQMNIKVKNMTLDEINNTYCNNDYYNQAIREEPIDLLDRSFSSSSWPF

SEQ ID NO: 9 - DNA, 52 bp, Artificial
  primer
ttaacggcccgggaaggagaatatacatatgacacgacaaaaagcaacgctc

SEQ ID NO: 10 - DNA, 27 bp, Artificial
  primer
tctgatcggtaccgaaatgagatataa

SEQ ID NO: 11 - DNA, 37 bp, Artificial
  primer
agagatttcccgggcaatatgttttcctttattttcc

(19) United States
(12) Patent Application Publication
(10) Pub. No.: US 2007/0166807 A1
(43) Pub. Date: Jul. 19, 2007

(54) Method for Producing Aromatic L-Amino Acid Using Bacterium Belonging to the Genus Methylophilus

(76) Inventors: Irina L'vovna Tokmakova, Moscow region (RU); Natalya Vasilievna Gorshkova, Moscow (RU); Elena Georgievna Abalakina, Moscow (RU); Yurgis Antanas Vladovich Iomantas, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/612,637

(22) Filed: Dec. 19, 2006

Related U.S. Application Data
(60) Provisional application No. 60/806,753, filed on Jul. 7, 2006.

(30) Foreign Application Priority Data
Dec. 27, 2005 (RU) 2005140649

(51) Int. Cl. C12P 13/04; C12P 13/24; C12P 13/22; C12N 15/74; C12N 1/21

(52) U.S. Cl. 435/106; 435/107; 435/108; 435/252.3; 435/471

(57) ABSTRACT

The present invention provides a method for producing an L-amino acid using a bacterium belonging to the genus Methylophilus which has been modified to enhance expression of genes involved in the efflux of aromatic L-amino acids, particularly the yddG, yddL, yddK, and yddJ genes from Escherichia coli.

The present application claims priority under 35 U.S.C. §119 to Russian Patent Application No. 2005140649, filed Dec. 27, 2005, and U.S. Provisional Patent Application No. 60/806,753, filed Jul. 7, 2006, the entirety of both of which is incorporated by reference. Also, the Sequence Listing filed electronically via EFS-Web herewith is hereby incorporated by reference (File name: US-250 Seq List; File size: 21 KB; Date recorded: Dec. 19, 2006).

### BACKGROUND OF THE INVENTION

1. Field of the Invention

The present invention relates to the microbiological industry, and specifically to a method for producing an aromatic L-amino acid, particularly L-phenylalanine, using a bacterium belonging to the genus Methylophilus, wherein said bacterium has been modified to enhance expression of genes encoding proteins which are involved in aromatic L-amino acid efflux.

2. Brief Description of the Related Art

L-Amino acids such as L-lysine, L-glutamic acid, L-threonine, L-leucine, L-isoleucine, L-valine, and L-phenylalanine are industrially produced by fermentation using microorganisms that belong to the genus Brevibacterium, Corynebacterium, Bacillus, Escherichia, Streptomyces, Pseudomonas, Arthrobacter, Serratia, Penicillium, Candida, and the like. In order to improve the productivity, strains isolated from nature or artificial mutants thereof have been used. Various techniques have been disclosed to increase the L-glutamic acid-producing ability by enhancing activities of L-glutamic acid biosynthetic enzymes by using recombinant DNA techniques.

The production of L-amino acids has been considerably increased by breeding microorganisms such as those mentioned above and improving production methods. However, in order to meet increased demand in the future, development of more efficient methods for producing L-amino acids at a lower cost is still desirable.

To produce amino acids by fermentation of methanol, which is a raw material available in large amounts at a low cost, conventionally known methods include using microorganisms that belong to the genus Achromobacter or Pseudomonas (Japanese Patent Publication (Kokoku) No. 45-25273/1970), Protaminobacter (Japanese Patent Application Laid-open (Kokai) No. 49-125590/1974), Protaminobacter or Methanomonas (Japanese Patent Application Laid-open (Kokai) No. 50-25790/1975), Microcyclus (Japanese Patent Application Laid-open (Kokai) No. 52-18886/1977), Methylobacillus (Japanese Patent Application Laid-open (Kokai) No. 4-91793/1992), Bacillus (Japanese Patent Application Laid-open (Kokai) No. 3-505284/1991), and so forth.

A method for producing L-phenylalanine by culturing a bacterium belonging to the genus Methylophilus which has an ability to produce L-phenylalanine and is resistant to a phenylalanine analog, such as DL-p-fluorophenylalanine, m-fluorophenylalanine and cinnamic acid, has been disclosed (EP1134283B1).

Also, a method for producing an L-amino acid, including L-phenylalanine, by culturing a microorganism having an ability to produce an L-amino acid in a medium, whereby the L-amino acid accumulates in the medium, and collecting the L-amino acid from the medium, whereby said microorganism is a methanol-utilizing bacterium having the Entner-Doudoroff pathway in which 6-phosphogluconate dehydratase activity and/or 2-keto-3-deoxy-6-phosphogluconate aldolase activity is enhanced, has been disclosed (US patent application 20040142435A1).

A method for producing an L-amino acid, including L-phenylalanine, by culturing a microorganism which has been modified so that expression of an ybjE gene is enhanced in a liquid medium containing methanol as a major carbon source has also been disclosed (PCT application WO05073390A2).

A process for producing an L-amino acid, for example L-phenylalanine or L-tryptophan, by culturing an Escherichia bacterium in a medium, the L-amino acid productivity of the bacterium has been enhanced by elevating the activity of a protein encoded by the yddG gene only has been disclosed (PCT application WO03044192A1).

But currently, there have been no reports of enhancing expression of the yddG, yddL, yddK and yddJ genes for the purpose of producing L-amino acids, such as L-phenylalanine, using methanol-utilizing bacteria belonging to the genus Methylophilus.

### SUMMARY OF THE INVENTION

Objects of the present invention include enhancing the productivity of aromatic L-amino acid-producing strains and providing a method for producing an aromatic L-amino acid using these strains.

The above objects were achieved by finding that enhancing or increasing expression of the yddG, yddL, yddK, and yddJ genes from E. coli in a bacterium belonging to the genus Methylophilus, particularly Methylophilus methylotrophus, can increase production of aromatic L-amino acids, such as L-phenylalanine, L-tryptophan, and L-tyrosine, preferably L-phenylalanine.

The present invention provides a bacterium belonging to the genus Methylophilus having an increased ability to produce aromatic L-amino acids, such as L-phenylalanine, L-tryptophan, and L-tyrosine, preferably L-phenylalanine.

It is an object of the present invention to provide a bacterium belonging to the genus Methylophilus having an ability to produce an aromatic L-amino acid, wherein said bacterium has been modified to increase expression of genes coding for proteins involved in aromatic L-amino acid efflux.

It is a further object of the present invention to provide the bacterium as described above, wherein said genes are obtained from a bacterium belonging to the genus Escherichia.

It is a further object of the present invention to provide the bacterium as described above, wherein said genes comprise yddG, yddL, yddK, and yddJ.

It is a further object of the present invention to provide the bacterium as described above, wherein said expression is increased by modifying an expression control sequence which controls expression of said genes.

It is a further object of the present invention to provide the bacterium as described above, wherein said expression is increased by increasing the copy number of said genes.

It is a further object of the present invention to provide the bacterium as described above, wherein said aromatic L-amino acid is selected from the group consisting of L-phenylalanine, L-tyrosine, and L-tryptophan.

It is a further object of the present invention to provide the bacterium as described above, wherein said aromatic L-amino acid is L-phenylalanine.

It is a further object of the present invention to provide the bacterium as described above, wherein said bacterium is Methylophilus methylotrophus.

It is a further object of the present invention to provide a method for producing an aromatic L-amino acid comprising:
cultivating the bacterium described above in a medium containing methanol, and
collecting said L-amino acid from the medium.

It is a further object of the present invention to provide the method as described above, wherein said aromatic L-amino acid is selected from the group consisting of L-phenylalanine, L-tyrosine, and L-tryptophan.

It is a further object of the present invention to provide the method as described above, wherein said aromatic L-amino acid is L-phenylalanine.

The present invention is described in detail below.

### BRIEF DESCRIPTION OF THE DRAWINGS

FIG. 1 shows the structure of PCR-amplified DNA fragment carrying RBS^{fmd} and the yddG, yddL, yddK, yddJ genes from E. coli encoding a complete set for the phenylalanine efflux pump.

FIG. 2 shows the structure of deletion derivatives carrying the yddG gene.

### DESCRIPTION OF THE PREFERRED EMBODIMENTS

1. Bacterium of the Present Invention

The bacterium of the present invention is a Methylophilus bacterium having an ability to produce the aromatic L-amino acid, wherein said bacterium has been modified to enhance or increase the expression of genes involved in aromatic L-amino acid efflux.

In the present invention, “bacterium having an ability to produce an aromatic L-amino acid” means a bacterium which has an ability to produce and excrete L-amino acid into a medium when the bacterium is cultured in the medium. The term “bacterium having ability to produce aromatic L-amino acid” as used herein also means a bacterium which is able to produce and cause accumulation of an L-amino acid in a culture medium in an amount larger than a wild-type or parental strain of M. methylotrophus, such as M. methylotrophus AS1, and preferably means that the microorganism is able to cause accumulation in a medium of an amount not less than 0.05 g/L, more preferably not less than 0.1 g/L, of the target L-amino acid. The term “aromatic L-amino acid” includes L-phenylalanine, L-tryptophan, and L-tyrosine. L-phenylalanine is particularly preferred.

The genus Methylophilus includes the following bacteria: Methylophilus freyburgensis, Methylophilus leisingeri, Methylophilus methylotrophus, Methylophilus quaylei etc. Specifically, those classified as Methylophilus according to the taxonomy used in the NCBI (National Center for Biotechnology Information) database can be used and are included. A bacterium belonging to the species Methylophilus methylotrophus is preferred. Specific examples of Methylophilus bacteria which may be used in the present invention include the Methylophilus methylotrophus strain AS1(NCIMB10515) and so forth. The Methylophilus methylotrophus strain AS1 (NCIMB10515) is available from the National Collections of Industrial and Marine Bacteria (Address NCIMB Lts., Torry Research Station, 135, Abbey Road, Aberdeen AB98DG, United Kingdom).

Genes coding for proteins involved in aromatic L-amino acid efflux include genes coding for proteins capable of pumping aromatic L-amino acids out of the bacterial cells. Increased activity of such proteins imparts resistance to aromatic L-amino acids of the bacteria and results in increasing accumulation of aromatic L-amino acids in the medium when such bacterium is cultivated in the medium.

The phrase “a bacterium belonging to the genus Escherichia” means that the bacterium is classified into the genus Escherichia according to the classification known to a person skilled in the art of microbiology. Examples of a bacterium belonging to the genus Escherichia as used in the present invention include, but are not limited to, Escherichia coli (E. coli).

The bacterium belonging to the genus Escherichia that can be used in the present invention is not particularly limited; however, e.g., bacteria described by Neidhardt, F. C. et al. (Escherichia coli and Salmonella typhimurium, American Society for Microbiology, Washington D.C., 1208, Table 1) are encompassed by the present invention.

The phrase “bacterium has been modified to increase expression of genes” means that the bacterium has been modified in such a way that the modified bacterium contains increased amounts of the proteins coded for by said genes, as compared with an unmodified bacterium.

The phrase “the enhanced or increased expression of genes” means that the expression level of the genes is higher than that of a non-modified strain, for example, a wild-type strain. Examples of such modifications include increasing the copy number of the expressed gene(s) per cell, and/or increasing the expression level of the gene(s) by modification of an adjacent region of the gene, including sequences controlling gene expression, such as promoters, enhancers, attenuators, ribosome-binding sites, etc., and so forth.

The phrase “genes coding for proteins involved in aromatic L-amino acid efflux” means that when culturing a microorganism which has been modified to enhance or increase expression of the genes, the amount of L-amino acid secreted into the medium by the microorganism is more than that of an L-amino acid secreted from a non-modified strain, such as a parent strain or a corresponding wild-type strain. The increase in L-amino acid-efflux ability is observed by determining the increase in concentration of the L-amino acid in the medium. Furthermore, the increase in L-amino acid-efflux ability is also observed by determining the decrease in intracellular concentration of the L-amino acid upon introduction of the gene into a microorganism. The amount of L-amino acid secreted from the microorganism of the present invention is preferably increased by 10% or more, more preferably 30% or more, particularly preferably 50% or more, when compared to the amount of L-amino acid exported from a non-modified strain. Furthermore, the increase in L-amino acid-efflux ability is also observed in terms of a decrease in intracellular concentration of the L-amino acid upon introduction of genes into a microorganism. For example, the intracellular concentration of an L-amino acid can be measured as follows: an appropriate amount of silicon oil having specific gravity of 1.07 is added to a medium containing microbial cells, and cells are collected from the medium by centrifugation, preferably at 12,000 rpm for 2 minutes. Then the cells are treated with 22% perchloric acid (Ishizaki, A. et al, Biotech. Techniq., 9, 6, 409 (1995)). Using cells prepared in this way, an intracellular concentration of an L-amino acid can be measured. Furthermore, “L-amino acid-efflux ability” can be examined indirectly by measuring cellular uptake of radiolabeled L-amino acid using everted membrane vesicles (Pittman, M. S. et al, J. Biol. Chem., 277, 51, 49841-49849 (2002)). For example, everted membrane vesicles are prepared from cells into which gene is introduced. Then, ATP or other substrates which provide driving energy are added to the vesicles, and cellular uptake of radiolabeled L-amino acid is measured. Alternatively, “L-amino acid-export ability” may be examined by measuring the rate of the exchange reaction between a non-labeled amino acid and a labeled amino acid in active cells.

The examples of genes from bacterium belonging to the genus Escherichia involved in aromatic L-amino acid efflux include the yddG, yddL, yddK and yddJ genes iosolated from, or native to E. coli.

The yddG gene encodes the putative transmembrane YddG protein (synonym—B1473). The yddG gene (nucleotides complementary to nucleotides positions 1544312 to 1545193; GenBank accession no. NC_{—}000913.2; gi:49175990; SEQ ID NO: 1) is located between the yddL and fdnG genes on the chromosome of E. coli K-12. The nucleotide sequence of the yddG gene and the amino acid sequence of the YddG protein encoded by the yddG gene are shown in SEQ ID NO: 1 and SEQ ID NO: 2, respectively.

The yddL gene encodes putative outer membrane porin YddL protein (synonym—B1472). The yddL gene (nucleotides complementary to nucleotides positions 1543762 to 1544052; GenBank accession no. NC_{—}000913.2; gi:49175990; SEQ ID NO: 3) is located between the yddG and yddK genes on the chromosome of E. coli K-12. The nucleotide sequence of the yddL gene and the amino acid sequence of the YddL protein encoded by the yddL gene are shown in SEQ ID NO: 3 and SEQ ID NO: 4, respectively.

The yddK gene encodes the putative glycoprotein YddK (synonym—B1471). The yddK gene (nucleotides complementary to nucleotides positions 1542782 to 1543738; GenBank accession no. NC_{—}000913.2; gi:49175990; SEQ ID NO: 5) is located between the yddJ and yddL genes on the chromosome of E. coli K-12. The nucleotide sequence of the yddK gene and the amino acid sequence of the YddK protein encoded by the yddK gene are shown in SEQ ID NO: 5 and SEQ ID NO: 6, respectively.

The yddJ gene encodes the YddJ protein (synonym—B1470), the function of which is unknown. The yddJ gene (nucleotides complementary to nucleotides positions 1542408 to 1542743; GenBank accession no. NC_{—}000913.2; gi:49175990; SEQ ID NO: 7) is located between the narU and yddK genes on the chromosome of E. coli K-12. The nucleotide sequence of the yddjgene and the amino acid sequence of the YddJ protein encoded by the yddJ gene are shown in SEQ ID NO: 7 and SEQ ID NO: 8, respectively.

Since there may be some differences in DNA sequences between Escherichia strains, the above-described genes to be overexpressed are not limited to the nucleotide sequences shown in SEQ ID NOS: 1, 3, 5 and 7, but may also include nucleotide sequences similar to those shown in SEQ ID NOS: 1, 3, 5 and 7. Therefore, the protein variants encoded by the above-described genes may have a similarity of not less than 80%, preferably not less than 90%, and most preferably not less than 95%, with respect to the entire amino acid sequences shown in SEQ ID NOS. 2, 4, 6 and 8, as long as the ability of the proteins to cause efflux of aromatic L-amino acids is maintained.

Moreover, the above-described genes may be represented by variants which can hybridize under stringent conditions with the nucleotide sequences shown in SEQ ID NOS: 1, 3, 5 and 7 or with probes prepared based on these nucleotide sequences, provided that they encode functional proteins. “Stringent conditions” include those under which a specific hybrid is formed and a non-specific hybrid is not formed. For example, stringent conditions are exemplified by washing at 60° C. one time, preferably two or three times, with a solution containing 1×SSC and 0.1% SDS, preferably 0.1×SSC and 0.1% SDS. The length of the probe may be suitably selected, depending on the hybridization conditions, and usually varies from 100 bp to 1 kbp.

Enhancement of gene expression may be achieved by increasing the copy number of the genes, and/or by introducing a vector carring the gene. The vector for Methylophilus bacteria, for example, is a plasmid that is autonomously replicable in cells of Methylophilus bacteria. Specific examples of vectors for Methylophilus bacteria include RSF1010 and derivatives thereof, such as pAYC32 (Chistorerdov, A. Y., Tsygankov, Y. D. Plasmid, 16, 161-167 (1986)), pMFY42 (Gene, 44, 53 (1990)), pRP301, and pTB70 (Nature, 287, 396, (1980)).

Increasing or enhancing gene expression may be achieved by introducing multiple copies of the gene into a bacterial chromosome by, for example, the method of homologous recombination, Mu integration, or the like. The copy number of an expressed gene can be estimated, for example, by restricting the chromosomal DNA followed by Southern blotting using a probe based on the gene sequence, by fluorescence in situ hybridization (FISH), and the like.

Increasing or enhancing gene expression may also be achieved by placing the gene(s) under the control of a potent promoter suitable for functioning in the bacterium belonging to the genus Methylophilus. For example, the lac promoter, the trp promoter, the trc promoter, the P_{R}, or the P_{L} promoters of lambda phage are known as potent promoters. Use of a potent promoter can be combined with multiplication of gene copies.

Alternatively, the effect of a promoter can be enhanced by, for example, introducing a mutation into the promoter to increase the transcription level of a gene located downstream of the promoter. Furthermore, it is known that substitution of several nucleotides in the spacer region between the ribosome binding site (RBS) and the start codon, especially the sequences immediately upstream of the start codon, can profoundly affect the mRNA translatability. For example, a 20-fold range in the expression levels was found, depending on the nature of the three nucleotides preceding the start codon (Gold et al., Annu. Rev. Microbiol., 35, 365-403, 1981; Hui et al., EMBO J., 3, 623-629, 1984). Previously, it was shown that the rhtA23 mutation is an A-for-G substitution at the −1 position relative to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457). Therefore, it may be suggested that the rhtA23 mutation enhances rhtA gene expression and, as a consequence, increases resistance to threonine, homoserine, and some other substances transported out of cells.

The level of gene expression can be estimated by measuring the amount of MRNA transcribed from the gene using various well-known methods, including Northern blotting, quantitative RT-PCR, and the like. The amount of the protein encoded by the gene can be measured by well-known methods, including SDS-PAGE followed by immunoblotting assay (Western blotting analysis) and the like.

Methods for preparation of plasmid DNA, digestion and ligation of DNA, transformation, selection of an oligonucleotide as a primer, and the like may be ordinary methods well-known to those skilled in the art. These methods are described, for instance, in Sambrook, J., Fritsch, E. F., and Maniatis, T., “Molecular Cloning: A Laboratory Manual, Second Edition”, Cold Spring Harbor Laboratory Press (1989)

L-Amino Acid-Producing Bacteria

The bacterium of the present invention can be obtained by enhancing expression of genes coding for proteins involved in aromatic L-amino acid efflux in a bacterium, which inherently has the ability to produce aromatic L-amino acids. Alternatively, the bacterium of present invention can be obtained by imparting the ability to produce aromatic L-amino acids to a bacterium already having the enhanced expression of genes coding for proteins involved in aromatic L-amino acid efflux.

The ability to produce aromatic amino acids may be an original ability of a wild-type strain of bacterium or an ability which has been imparted by breeding.

Hereinafter, methods for imparting an aromatic amino acid-producing ability to a parent strain as mentioned above will be explained.

In order to impart aromatic amino acid-producing ability, methods conventionally used for breeding an L-amino acid-producing bacterium belonging to the genus Escherichia or Coryneform bacterium and so forth can be used. For example, methods for obtaining an auxotrophic mutant strain, analogue-resistant strain, or metabolic regulation mutant strain having L-amino acid-producing ability, and methods for creating a recombinant strain having enhanced activity of an L-amino acid-biosynthetic enzyme can be used (“Amino Acid Fermentation”, the Japan Scientific Societies Press [Gakkai Shuppan Center], 1st Edition, published on May 30, 1986, pp. 77-100). When breeding L-amino acid-producing bacteria using these methods, one or more properties, including auxotrophy, analogue resistance, and metabolic regulation mutation, may be imparted.

When a recombinant strain is created, the activity of single or multiple L-amino acid-biosynthetic enzymes may be enhanced. Furthermore, methods imparting properties of auxotrophy, analogue resistance, and metabolic regulation mutation may be combined with methods enhancing an activity of L-amino acid-biosynthetic enzyme.

An auxotrophic mutant strain, L-amino acid analogue-resistant strain, or metabolic regulation-mutated strain having an L-amino acid-producing ability can be obtained by subjecting a parent or wild-type strain to a typical mutagenesis treatment such as X-ray or ultraviolet ray irradiation, treatment with a mutagenesis agent such as N-methyl-N′-nitro-N-nitrosoguanidine (NTG). Then, an auxotrophic strain, analogue-resistant strain or metabolic regulation mutant strain which has an L-amino acid-producing ability may be selected from the mutated strains.

Bacteria having an ability to produce aromatic amino acids can be obtained by enhancing the activities of 3-deoxy-D-arabinoheptulosonate-7-phosphate synthase (encoded by aroG gene), 3-dehydroquinate synthase (encoded by aroB gene), shikimate kinase (encoded by aroL gene), chorismate mutase (encoded by aroA gene), chorismate synthase (encoded by aroC gene) (PCT application WO02/38777).

2. Method of the Present Invention

The method of the present invention is a method for producing an L-amino acid by cultivating the bacterium of the present invention in a culture medium to produce and excrete the L-amino acid into the medium, and collecting the L-amino acid from the medium.

In the present invention, the cultivation, collection, and purification of L-amino acid from the medium and the like may be performed in a manner similar to conventional fermentation methods wherein an amino acid is produced using a bacterium.

A medium used for culture may be either a synthetic or natural medium, so long as the medium includes a carbon source and a nitrogen source and minerals and, if necessary, appropriate amounts of nutrients which the bacterium requires for growth. The carbon source for bacterium belonging to the genus Methylophilus includes C1 compounds, such as methanol. If methanol is used as the main carbon source, aromatic L-amino acids, such as L-phenylalanine, can be produced at low cost. When methanol is used as the main carbon source, it is added to a medium in an amount of 0.001 to 30%. As a nitrogen source, various ammonium salts such as ammonia and ammonium sulfate, other nitrogen compounds such as amines, a natural nitrogen source such as peptone, soybean-hydrolysate, and digested fermentative microorganisms can be used. As minerals, potassium monophosphate, magnesium sulfate, sodium chloride, ferrous sulfate, manganese sulfate, calcium chloride, and the like can be used. As vitamins, thiamine, yeast extract, and the like, can be used.

The cultivation is preferably performed under aerobic conditions, such as a shaking culture, and a stirring culture with aeration, at the temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to accumulation of the target L-amino acid in the liquid medium.

After cultivation, solids, such as cells, can be removed from the liquid medium by centrifugation or membrane filtration, and then the L-amino acid can be collected and purified by ion-exchange, concentration, and/or crystallization methods.

### EXAMPLES

The present invention will be more concretely explained below with reference to the following non-limiting Examples.

### Example 1

### The Cloning, Analysis, and Application of the L-Phenylalanine Efflux Pump Genes from E. coli in M. methylotrophus Strains

It has been reported that enhancing the activity of a protein encoded by the yddG gene from E. coli increases production of L-amino acid, for example L-phenylalanine or L-tryptophan, by E. coli bacterium when the bacterium is cultured in a medium (PCT application WO03044192A1).

Therefore, it was supposed that the yddG gene encodes an L-phenylalanine efflux pump.

The M. methylotrophus is a good test microorganism for analysis of L-phenylalanine efflux genes, because it does not grow on a medium containing a high L-phenylalanine concentration (2.0-5.0 g/L). And the active L-phenylalanine efflux pump imparts resistance to phenylalanine to a M. methylotrophus bacterium.

At first, a single yddG gene from E. coli was amplified by PCR. It was amplified with the new ribosome binding site, AGGAGA, of M. methylotrophus formamidase (fmd) gene (Mills, J. et al, Eur. J. Biochem., 251, p. 45-53 (1998)) using two synthetic primers P1 (SEQ ID NO: 9) and P2 (SEQ ID NO: 10). Primer P1 contains a SmaI restriction site and RBS of M. methylotrophus formamidase gene. Primer P2 contains a KpnI restriction site. The chromosomal DNA of E. coli strain MG1655 was used as a template.

The resulting amplified DNA fragment was treated with SmaI and KpnI restictases and ligated under the control of the P_{tac} promoter into the pTACTER2 vector which had been previously treated with the same restricases. The resulting plasmid was designated as pTYD6 (pTACTER2-Ptac^{Eco}-RBS^{fmd}-yddG^{Eco}).

The pTACTER2 vector was obtained from the pAYCTER3 vector by introducing a P_{tac} promoter. For that purpose, the commercially available P_{tac} promoter (Pharmacia, Sweden) was treated with HindIII and BamHI restrictases and ligated into a pAYCTER3 vector which had been previously treated with the same restricases.

The pAYCTER3 vector is a derivative of pAYC32, a moderate copy number and very stable vector constructed on the basis of plasmid RSF1010 (Christoserdov A. Y., Tsygankov Y. D, Broad-host range vectors derived from a RSF 1010 Tnl plasmid, Plasmid, 1986, v. 16, pp. 161-167). The pAYCTER3 vector was obtained by introduction of the polylinker from the pUC19 plasmid and the strong terminator rrnB into the pAYC32 plasmid instead of its native terminator. Detailed construction of pAYCTER3 vector is described in the (PCT application WO03044192A1).

Then, the resulting pTYD6 plasmid was mobilized from E. coli strain S17-1 (McPheat, W. L., FEMS Microbiology Letters, 41, 185-188 (1987)) into M. methylotrophus strain AS1. Methylophilus methylotropus strain AS1 is available from National Collections of Industrial and Marine Bacteria (Address NCIMB Lts., Torry Research Station, 135, Abbey Road, Aberdeen AB98DG, United Kingdom).

Both strains AS1 and the resulting AS1/pTYD6 were tested by fermenting in tubes in 121 medium with 2% methanol (conditions for fermentation and method for measurement of L-phenylalanine concentration: see Example 2 below). The fermentation data are presented in Table 1. It can be seen, the wild-type M. methylotrophus strain AS1 did not produce phenylalanine, when only the yddG gene from E. coli was amplified on the plasmid.

It was supposed that a single yddG gene from E.coli encodes only an inner membrane protein which is insufficient for functioning as an exporter across the inner and outer membranes in the M. methylotrophus strain. Thus, in order to form a multicomponent phenylalanine efflux pump in M. methylotrophus bacteria, it was necessary to find and clone other genes from E. coli besides the yddG gene.

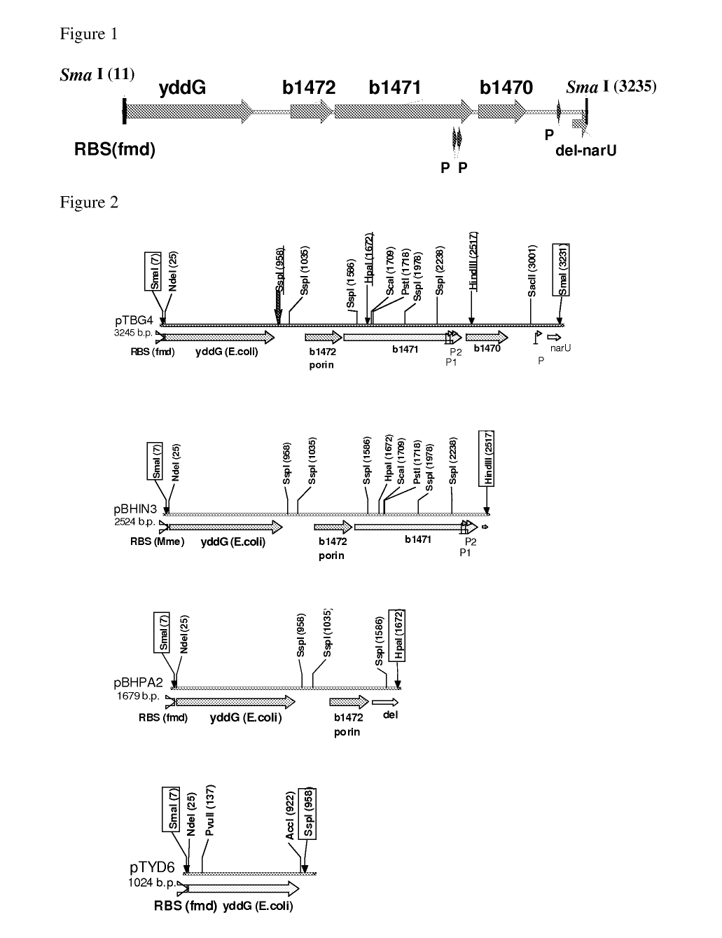

Genes located around the yddG gene in the E. coli genome were analyzed and it was found that several downstream genes encoded proteins homologous to outer membrane proteins (porins) in other bacteria. This group of E. coli genes, yddG, yddL, yddK and yddJ (FIG. 1), was amplified by PCR, cloned into pTACTER2 under the control of the P_{tac} promoter and RBS of formamidase (fmd) gene, and transferred into M. methylotrophus strain AS1.

For that purpose, the group of genes, yddG, yddL, yddK and yddJ, was amplified by PCR using primer P1 (SEQ ID NO: 9) and P3 (SEQ ID NO: 11). Primer P3 contains SmaI restriction site.

The resulting amplified DNA fragment was treated with SmaI restrictase and ligated under the control of the P_{tac} promoter and RBS of fmd gene into pTACTER2 vector which had been previously treated with the same restrictase. The resulting plasmid was designated as pTBG4 (pAYCTER3-P_{tac}^{Eco}-RBS^{Mme}-yddG^{Eco}-yddL^{Eco}-yddG^{Eco}-yddJ^{Eco}). Then, the resulting pTBG4 plasmid was mobilized from E. coli strain S17-1 ((McPheat, W. L., FEMS Microbiology Letters, 41, 185-188 (1987)) into M. methylotrophus strain AS1.

The wild-type M. methylotrophus strain AS1 is sensitive to high concentrations of L-phenylalanine and did not grow in medium containing 1 g/L or more of L-phenylalanine. The L-phenylalanine inhibits a single DAHP synthase. This feature helps to elucidate the E. coli genes which encode the complete set of proteins for the phenylalanine efflux pump machinery functioning in the M. methylotrophus strain. The active E. coli L-phenylalanine efflux machinery exports L-phenylalanine from M. methylotrophus, reduces inner concentration of L-phenylalanine and allows bacterium to grow on medium containing L-phenylalanine in high concentration.

Several strains were tested for resistance to L-phenylalanine. These are M. methylotrophus strain AS1/pTBG4 (pAYCTER3-P_{tac}^{Eco}-RBS^{Mme}-yddG^{Eco}-yddL^{Eco}-yddK^{Eco}-yddJ^{Eco}) (clones No. 7, No. 8, No. 9); AS1/pTYD6 (pAYCTER3-P_{tac}^{Eco}-RBSM^{Mme}-yddG^{Eco}); AS1/pAYCTER3. The phenylalanine sensitivity/resistance experiment was performed under a standard scheme. The strains were grown overnight in liquid medium, then diluted to 10⁵ cells/ml and plated on 121-medium agar plates containing 1% methanol, 100 mg/L ampicillin, and different concentrations of L-phenylalanine (g/L). The colony growth was analyzed after 72 hours. Data from the experiment are presented in Table 3.

It was determined that only the M. methylotrophus AS1/pTBG4 strain carrying the cloned four yddG-yddL-yddK -yddJ genes from E. coli can grow in a medium containing a high concentration of phenylalanine, such as 5 g/L, 2.5 g/L or 1.2 g/L.

The M. methylotrophus AS1/pTYD6 strain carrying a single yddG gene did not grow in a medium containing a high concentration of phenylalanine (1.2, 2.5, 5 g/L) similar to the control strain AS1/pAYCTER3.

Moreover, clone No. 8 of M. methylotrophus AS1/pTBG4 strain grew poorly (leaky growth) in medium without L-phenylalanine. This can mean that the active E. coli phenylalanine efflux system (yddG-yddL-yddK -yddj) really functions in the M. methylotrophus strain AS1 and generates leaky auxotrophy if grown in medium without L-phenylalanine.

Production of L-phenylalanine by strain AS1 and three clones of strain AS1/pTBG4 were tested by fermenting in tubes 121 medium with 2% methanol (see the Reference example below for conditions for fermentation and the method for measuring the L-phenylalanine concentration). Data from the fermentation are presented in Table 2. It can be seen that the wild-type M. methylotrophus strain AS1 carrying the plasmid with an active phenylalanine efflux system secreted between 0.06-0.11 g/L of L-phenylalanine into the medium.

To support the statement that all four E. coli genes are necessary for normal functioning of the phenylalanine efflux system in M. methylotrophus, two pTBG4 deletion derivatives carrying two and three Phe-efflux genes were constructed using suitable restrictases (FIG. 2) and resistance of strains AS1 carrying such derivatives to L-phenylalanine and production of L-phenylalanine by such strains were tested.

The HpaI-SmaI deletion derivative of pTBG4 plasmid, pBHPA2 (pAYCTER3-P_{tac}-RBS^{fmd}-yddG^{Eco}-yddL^{Eco}), had only two E. coli genes and did not impart resistance to L-phenylalanine. The strain AS1 carrying that derivative did not grow in medium containing 3 g/L L-phenylalanine and did not secrete any L-phenylalanine to the medium. This means that the L-phenylalanine efflux machinery was not complete.

The HindIII-deletion derivative of pTBG4 plasmid, pBHIN3 (pAYCTER3-P_{tac}-RBS^{fmd}-yddG^{Eco}-yddL^{Eco}-yddK^{Eco}), had three E. coli genes. The strain AS1 carrying that derivative also did not grow in medium containing 3 g/L L-phenylalanine and did not secrete any L-phenylalanine into the medium. This result means that the all four E. coli genes, yddG, yddL, yddK and yddJ, are necessary for secretion of L-phenylalanine from the M. methylotrophus strains.

### Example 2

### Fermentation Procedure for M. methylotrophus Strains and Measurement of L-Phenylalanine Concentration

1. Medium Composition and Procedure for Test Tube and Flask Cultivation

The cultivation of M. methylotrophus strains was carried out at 30° C. in 5 ml of 121 (Fe2, Fe3) medium containing 2% methanol and 3% calcium carbonate in tubes (18 mm diameter) 48-72 hours at 240 rpm on shaker.

The composition of 121(Fe2, Fe3) medium (1 liter):

| | | |
|---|---|---|
| K₂HPO₄ × 3H₂0 | 1.57 | g |
| KH₂PO₄ | 0.62 | g |
| (NH₄)₂SO₄ | 3 | g |
| NaCl | 0.1 | g |
| MgSO₄ × 7H₂0 | 0.2 | g |
| CaCl₂ | 0.025 | g |
| EDTA-Na₂ | 5 | mg |
| FeSO₄ × 7H₂0 | 3 | mg |
| FeCl₃ × 6H₂0 | 10 | mg |
| MnSO₄ × 5H₂0 | 0.01 | mg |
| ZnSO₄ × 7H₂0 | 0.07 | mg |
| Na₂MoO₄ × 2H₂0 | 0.01 | mg |
| H₃BO₃ | 0.01 | mg |
| CoCl₂ × 6H₂0 | 0.005 | mg |
| CuSO₄ × 5H₂0 | 0.005 | mg |
| Methanol | 20 | ml (filter sterilized) |
| CaCO₃ | 30 | g |

pH 7.0. Initial volume of medium in fermentation tubes: 5 ml.

Seed culture was grown in 5 ml of the same liquid medium without CaCO₃ in tubes 24 hours on shaker at 240 rpm at 30° C. Inoculums size—10%. Temperature was maintained at 30° C. Agitation—240 rpm on shaker.

2. Measurement of L-phenylalanine Concentration.

The L-phenylalanine concentration was measured by a fluorometric method described by, for example, McCaman, M. W. and Robins, E. (J. Lab. Clin. Med., 59, No. 5, 885-890 (1962)). Phenylalanine reacts with ninhydrin in the presence of copper ions to form a highly fluorescent complex. The fluorescence is greatly enhanced by inclusion of L-Leu-L-Ala dipeptide in the reaction mixture. The intensity of fluorescence measured in a flourometer is proportional to the phenylalanine concentration.

While the invention has been described in detail with reference to preferred embodiments thereof, it will be apparent to one skilled in the art that various changes can be made, and equivalents employed, without departing from the scope of the invention. All the cited references herein are incorporated as a part of this application by reference.

TABLE 1

| Strain | Cloned gene | Phe, g/L |
|---|---|---|
| AS1 | — | 0.02 |
| AS1/pTYD6 | P_{tac}^{Eco} − RBS^{Mme} − yddG^{Eco} | 0.02 |

TABLE 2

| | Growth in the presence | | | | | | |
|---|---|---|---|---|---|---|---|
| | of L-phenylalanine, g/l | | | | | | |
| Strain and cloned genes | 0 | 0.3 | 0.6 | 1.2 | 2.5 | 5 | 10 |
| AS1/pAYCTER3 | + | + | + | − | − | − | − |
| AS1/pTYD6 (yddG) | + | + | + | − | − | − | − |
| AS1/pTBG4 No. 7 | + | + | + | + | ++ | ++ | − |
| (yddG + b1472 + b1471 + b1470) | | | | | | | |
| AS1/pTBG4 No. 8 | +/− | +/− | + | + | ++ | ++ | − |
| (yddG + b1472 + b1471 + b1470) | | | | | | | |
| AS1/pTBG4 No. 9 | + | + | + | + | + | + | − |
| (yddG + b1472 + b1471 + b1470) | | | | | | | |

+/− is a leaky growth mini colony;
+ is normal growth;
++ is good growth

TABLE 3

| Strain | Cloned genes | Phe, g/L |
|---|---|---|
| AS1 | — | 0.03 |
| AS1/pAYCTER3 | — | 0.05 |
| AS1/pTBG4 No. 7 | P_{tac} − RBS^{fmd} − yddG + | 0.06 |
| | yddL + yddK + yddJ | |
| AS1/pTBG4 No. 9 | P_{tac} − RBS^{fmd} − yddG + | 0.09 |
| | yddL + yddK + yddJ | |
| AS1/pTBG4 No. 8 | P_{tac} − RBS^{fmd} − yddG + | 0.11 |
| (leaky) | yddL + yddK + yddJ | |

What is claimed is:

1. A Methylophilus bacterium which has the ability to produce aromatic L-amino acids, wherein said bacterium has been modified to increase expression of genes coding for proteins involved in aromatic L-amino acid efflux.

2. The bacterium according to claim 1, wherein said genes are obtained from an Escherichia bacterium.

3. The bacterium according to claim 2, wherein said genes comprise yddG, yddL, yddk, and yddJ.

4. The bacterium according to claim 1, wherein said expression is increased by modifying an expression control sequence which controls expression of said genes.

5. The bacterium according to claim 1, wherein said expression is increased by increasing the copy numbers of said genes.

6. The bacterium according to claim 1, wherein said aromatic L-amino acid is selected from the group consisting of L-phenylalanine, L-tyrosine, and L-tryptophan.

7. The bacterium according to claim 6, wherein said aromatic L-amino acid is L-phenylalanine.

8. The bacterium according to claim 1, wherein said bacterium is Methylophilus methylotrophus.

9. A method for producing an aromatic L-amino acid comprising:
cultivating the bacterium according to claim 1 in a medium containing methanol, and
collecting said L-amino acid from the medium.

10. The method according to claim 9, wherein said aromatic L-amino acid is selected from the group consisting of L-phenylalanine, L-tyrosine, and L-tryptophan.

11. The method according to claim 10, wherein said aromatic L-amino acid is L-phenylalanine.
